(19) United States
(12) Patent Application Publication
(10) Pub. No.: US 2003/0157639 A1
(43) Pub. Date: Aug. 21, 2003

(54) Modified chemokine receptor ccr-3 and assay

(76) Inventors: Ashley Antony Barnes, Stevenage (GB); Neil James Fraser, Stevenage (GB); Celestine Theresa O'Shaughnessy, Stevenage (GB); Alan Wise, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/276,950

(22) PCT Filed: May 31, 2001
(86) PCT No.: PCT/EP01/06195

(30) Foreign Application Priority Data
Jun. 1, 2000 (GB) 0013345.4

(51) Int. Cl. C12P 21/02; C12N 5/06; C07K 14/705; C07H 21/04

(52) U.S. Cl. 435/69.1; 435/320.1; 435/325; 530/350; 536/23.5

(57) ABSTRACT

The present invention relates to the modification of the CC chemokine receptor 3 to stabilise or enhance surface expression, for example, to reduce internalization and/or kinase-mediated phosphorylation. The invention also relates to assays for CCR3 receptor activity in which internalization and/or kinase mediated phosphorylation of the receptor is reduced.

### FIELD OF THE INVENTION

The invention relates to CC chemokine receptor 3 and modification of the receptor to stabilise or enhance surface expression, for example, to reduce internalization and/or kinase-mediated phosphorylation. The invention also relates to assays for CCR3 receptor activity in which internalization and/or kinase mediated phosphorylation of the receptor is reduced.

### BACKGROUND OF THE INVENTION

CC chemokine receptor 3 (CCR3) is a major receptor involved in regulating eosinophil trafficking. It is important to understand the biological and pathological properties of eosinophils since these cells cause proinflammatory effects in a number of diseases such as asthma, parasitic infections and malignancies. In addition to expression in eosinophils, CCR3 is expressed in other cell types implicated in inflammation such as T cells, Th2 lymphocytes and basophils including CNS microglia and neurons.

CCR3 binds multiple ligands including eotaxin, eotaxin-2, eotaxin-3, RANTES, MCP-2, MCP-3 and MCP-4. CCR3 is a G protein coupled receptor and its effects may therefore be mediated through binding to G-protein.

G-protein coupled receptors (GPCR) mediate cellular responses via activation of G-protein following ligand binding. In general, the cellular responses are rapidly attenuated. A variety of mechanisms may be responsible for this attenuation, including receptor desensitization, endocytosis and down regulation.

It is desirable to assay for agents which modulate CCR3-mediated cellular responses. In particular, it is desired to design high throughput assays. It is desirable to express CCR3 in heterologous cell systems in view of difficulties which may arise in routine isolation eosinophils for use in assays. However hitherto it has generally proved difficult to obtain consistent and adequate expression of the CCR3 receptor (especially the human receptor) in heterologous mammalian systems such as chinese hamster ovary (CHO) cells.

### SUMMARY OF THE INVENTION

The present invention provides a modified CCR3 receptor. In particular, a modified CCR3 receptor is provided which has been modified to enhance or stabilise expression of the receptor in a cell membrane, especially in mammalian cell membranes.

In preferred embodiments the modification is mutation or deletion of at least one phosphorylation site in the C-terminal region of the CCR3 receptor, mutation or deletion of the most C-terminal phosphorylation site of CCR3 receptor or mutation or deletion of the internalization site YIPF at position 327-330 of SEQ ID No.1 or an equivalent position of a variant CCR3 receptor, or addition of a suitable N-terminal signal sequence. In the most preferred embodiments, the modification is of a huamn CCR3 receptor (such as that shown in SEQ ID No. 1).

The invention also provides a polynucleotide encoding a modified CCR3 receptor according to the invention, an expression vector comprising such a polynucleotide and a host cell transformed with a polynucleotide or an expression vector of the invention.

The invention also provides an assay for investigating one or more properties of a CCR3 -receptor using a modified receptor according to the invention; performing the assay using cells expressing a modified receptor of the invention and investigating thereby one or more properties of the CCR3 receptor. Preferably the assay is for identification of modulators of eotaxin-mediated CCR3-receptor activity wherein the assay comprises contacting a compound under investigation with the cell and determining thereby whether a test compound modulates eotaxin-mediated CCR3 receptor binding or functional activity.

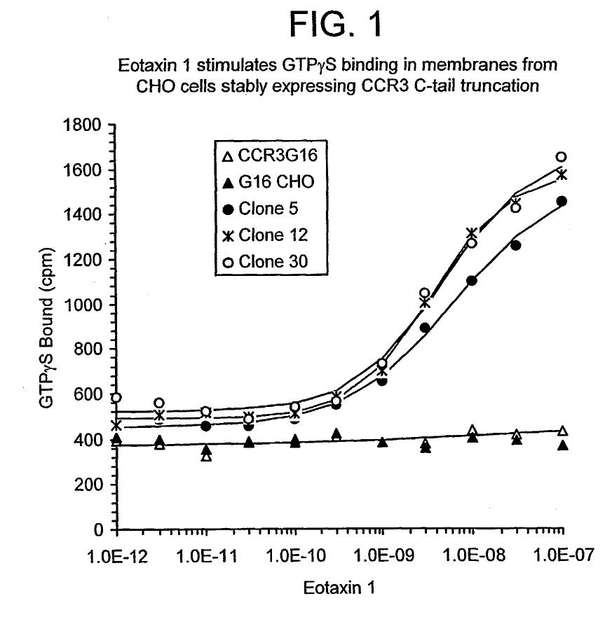

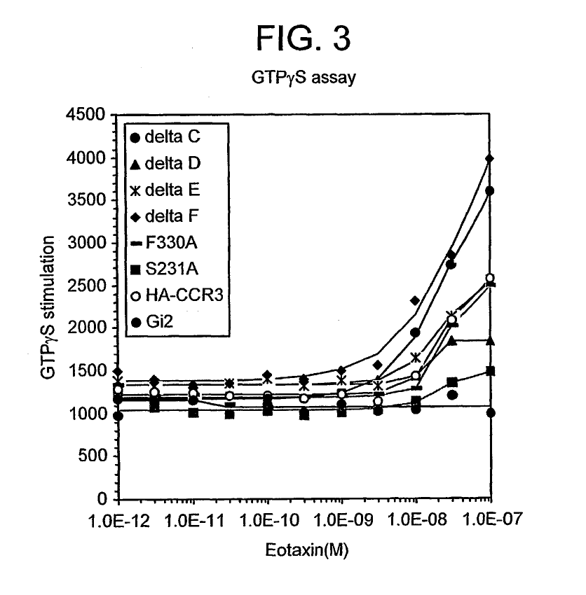

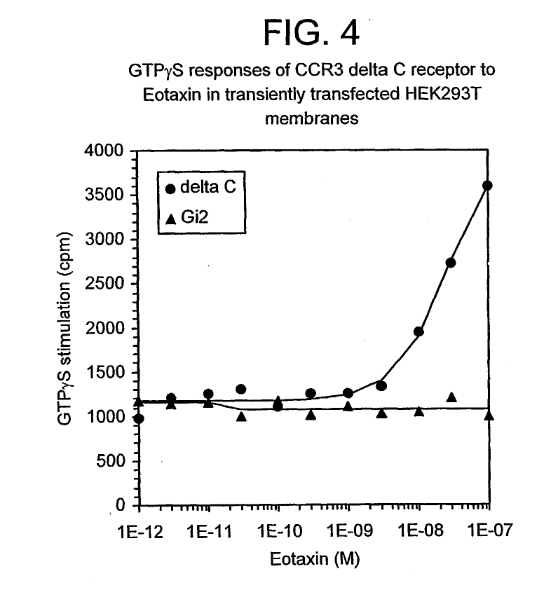

### DESCRIPTION OF THE FIGURES FIGS. 1, 2, 3 and 4 represent GTPγS activity in response to eotaxin in a cell line expressing various modified CCR3 receptors of the invention.

### DETAILED DESCRIPTION OF THE INVENTION

The invention relates to a modified CC chemokine receptor 3 (CCR3). The receptor is modified such that expression of the receptor in the cell membrane is stabilised and/or increased. The stabilisation or enhanced expression may be, for example, due to a reduction in internalization or phosphorylation of the intracellular portions of the receptor such that the receptor is retained in the cell membrane for a longer period of time or due to an increase in cell surface trafficking. Such a modified receptor may be expressed in cells and subsequently used in assays. In view of the reduction in internalization or phosphorylation of the receptor, cells expressing the receptor have a longer shelf life for use in assays.

The amino acid sequence for CCR3 is set out in SEQ ID NO. 1. SEQ ID NO.2 sets out the encoding polynucleotide sequence for this receptor. References to CCR3 relate to a CCR3 receptor having the sequence of SEQ ID NO.1 and variants thereof which retain the same function as naturally occurring CCR3. The invention thus includes modification of a CCR3 receptor which is a naturally occurring variant of SEQ ID NO.1 such as the equivalent receptor derived from a different animal species or different alleles shown by individuals of the same species.

A modified receptor of the present invention is modified to stabilise or enhance expression of the receptor and maintain the receptor in a cell membrane for a longer period of time when compared to wild type receptor. The modification may comprise deletion or mutation of at least one phosphorylation site, for example a sequence recognised by a kinase. Phosphorylation sites present in the C-terminus of CCR3 are located at positions 333, 339, 340, 341, 343, 345, 346 and 353 of SEQ ID No. 1. The modification of the phosphorylation site may comprise deletion of a phosphorylation site from the C-terminal region for example by deleting part or the whole of the C-terminal region spanning one or more of the phosphorylation sites. Alternatively, a mutation may be introduced into the C-terminal region such that phosphorylation will no longer occur through lack of recognition of the mutated phosphorylation site by kinases. In a preferred embodiment, at least the very end terminal phosphorylation site of the C-terminal is deleted, for example, through deletion of the C-terminal region from position 352 of SEQ ID No. 1.

In an alternative embodiment, the receptor is modified by deletion or mutation of the internalization sequence to reduce the activity of the sequence and thus reduce internalization of the receptor from the cell membrane. Again, the modification may comprise deletion of a portion of the C-terminal region which spans the internalization sequence. The internalization sequence of SEQ ID No. 1 has the sequence YIPF and is located at position 327-330 of SEQ ID No. 1. Alternatively, the whole of the C-terminus may be deleted up to and including the internalization sequence. For example, the deletion may comprise deletion of the C-terminus from at least amino acid residue 327 of SEQ ID No. 1 or a deletion from a position N-terminal to position 327.

In an alternative embodiment, a mutation is introduced into the internalization sequence. Preferably, the substituted amino acid introduced through the mutation is a non-conservative substitution such that the activity of the internalization sequence is reduced.

In an alternative embodiment, a signal sequence is provided. Suitable signal sequences include a signal sequence from type IIIa membrane protein. Alternatively, the amino acid structure of eukaryotic signal sequences and the point at which they are cleaved from mature proteins has been described in detail elsewhere (e.g. von Heijne (1986) Nuc. Acids. Res. 14 4683-4691, von Heijne et al. (1997) Protein Engineering 10 1-6). They are usually composed of about 20 amino acid residues (13->30) with a basic, polar amino terminus and an apolar core domain. In other embodiments the signal sequence could be one based on the predictions described by von Heijne et al. In a preferred aspect, the signal sequence is that from the human T-cell surface glycoprotein CD8 alpha chain precursor (T-cell differentiation antigen T8/leu-2) (SwissProt entry P01732) which has the amino acid sequence MALPVTALLLPLALLLHAARP to enhance translocation and production of a functional CCR3 receptor.

In preferred embodiments, at least one phosphorylation site and the internalization site are modified by deletion or substitution. In a preferred embodiment, the entire C-terminus of the naturally occurring protein is deleted.

Equivalent internalization sites or phosphorylation sites can be identified in variant proteins-by alignment of such equivalent sequences with the sequence of SEQ ID NO. 1 to identify the sites equivalent to the internalization site or phosphorylation site SEQ ID No. 1. Those skilled in the art will be readily able to identify such internalization or phosphorylation sites.

In a preferred aspect of the invention, the modification comprises removal of at least one phosphorylation site. In a preferred embodiment, the invention comprises deletion of both an internalization site and a phosphorylation site.

The polypeptides are provided in isolated form. The term “isolated” is intended to convey that the polypeptide is not in its native state, insofar as it has been purified at least to some extent or has been synthetically produced, for example by recombinant methods. The polypeptide may be mixed with carriers or diluents which will not interfere with the intended purpose of the polypeptide and still be regarded as substantially isolated. The term “isolated” therefore includes the possibility of the polypeptide being in combination with other biological or non-biological material, such as cells, suspensions of cells or cell fragments, proteins, peptides, expression vectors, organic or inorganic solvents, or other materials where appropriate, but excludes the situation where the polypeptide is in a state as found in nature.

A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press (1989), the disclosure of which is included herein in its entirety by way of reference.

A modified receptor of the present invention may also include a modified version of a variant of CCR3. Such a variant maintains the same functional characteristics of CCR3 other than the alterations in the stability of the receptor in a cell membrane.

Typically, polypeptides having at least 65% identity, preferably at least 80% or at least 90% identity and particularly preferably at least 95%, at least 97% or at least 99% identity, with the amino acid sequences of SEQ ID NO: 2 are considered as variant polypeptides. Such variants may include allelic variants and the deletion, modification or addition of single amino acids or groups of amino acids within the protein sequence, as long as the peptide maintains the basic biological functionality of the CCR3.

Amino acid substitutions may be made, for example from 1, 2 or 3 to 10, 20 or 30 substitutions. The modified polypeptide generally retains activity as a CCR3. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

Also included within the definition of CCR3 in accordance with the present invention are fragments of naturally occurring CCR3 or a variant thereof which maintain the ligand binding characteristics of CCR3. For example, the transmembrane regions of CCR3 may be replaced by transmembrane regions derived from another 7TM receptor for example another chemokine receptor such as CCR1, and thus comprises a chimeric receptor. A variant receptor is also one, for example in which transmembrane domain or domains have been deleted such as a five transmembrane domain CCR3 (see, for example, Ling et al., 1999, PNAS, 97, 7922).

In a particularly preferred embodiment of the invention, the polypeptide is expressed together with a signal sequence to promote its insertion into the cell membrane.

The modified CCR3 receptor retains the function of the naturally occurring CCR3 receptor except for a reduction in the activity of the internalization site or a phosphorylation site. In particular, it is preferred that the CCR3 receptor retains the ability to bind to at least one of the ligands selected from eotaxin, eotaxin-2, eotaxin-3, RANTES, MCP-2, MCP-3 and MCP-4. A modified receptor of the invention retains the ability to modulate a cellular response in response to ligand binding through. G protein coupling. Thus, a modified receptor according to the invention retains the ability to bind a ligand and couple to G protein to mediate a cellular response.

The invention also provides an isolated polynucleotide encoding a modified CCR3 receptor of the present invention.

The polynucleotide of the invention is generally capable of hybridizing selectively with a polynucleotide comprising all or part of the CCR3 gene. Thus, it may be capable of selectively hybridizing with all or part of the sequence shown in SEQ ID NO.2.

Selective hybridization means that generally the polynucleotide can hybridize to the gene region sequence at a level significantly above background. The signal level generated by the interaction between a polynucleotide of the invention and the gene region sequence is typically at least 10 fold, preferably at least 100 fold, as intense as interactions between other polynucleotides and the gene region sequence. The intensity of interaction may be measured, for example, by radiolabelling the polynucleotide, e.g. with 32p. Selective hybridization is typically achieved using conditions of medium to high stringency (for example 0.03M sodium chloride and 0.003M sodium citrate at from about 50° C. to about 60° C.).

Polynucleotides of the invention may comprise DNA or RNA. The polynucleotides may be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art.

The protein of the invention can be encoded by a polynucleotide of the invention. The protein may comprise all or part of a polypeptide sequence encoded by any of the polynucleotides represented by SEQ ID NOS: 1 or 2, or be a homologue of all or part of such a sequence.

Homologues of polynucleotide or protein sequences are referred to herein. Such homologues typically have at least 70% homology, preferably at least 80, 90%, 95%, 97% or 99% homology, for example over a region of at least 15, 20, 30, 100 more contiguous nucleotides or amino acids. The homology may calculated on the basis of amino acid identity (sometimes referred to as “hard homology”).

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (PN)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Polynucleotides or proteins of the invention may carry a revealing label. Suitable labels include radioisotopes such as ³²p or 35S, fluorescent labels, enzyme labels or other protein labels such as biotin.

Polynucleotides of the invention can be incorporated into a vector. Typically such a vector is a polynucleotide in which the sequence of the polynucleotide of the invention is present. The vector may be recombinant replicable vector, which may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

The vector may be an expression vector. In such a vector the polynucleotide of the invention in the vector is typically operably linked to a control sequence which is capable of providing for the expression of the coding sequence by the host cell.

The host cell is preferably a mammalian host cell such as CHO or HEK.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable host cell as described above to provide for expression of the protein of the invention. Thus, in a further aspect the invention provides a process for preparing the protein of the invention, which process comprises cultivating a host cell transformed or transfected with an expression vector as described above under conditions to provide for expression of the protein. Such cells are useful in the assays of the invention.

The vectors may be for example, plasmid, virus or phage vectors provided with an origin of replication, optionally a promoter for the expression of the said polynucleotide and optionally a regulator of the promoter. The vectors may contain one or more selectable marker genes.

Promoters and other expression regulation signals may be selected to be compatible with the host cell for which the expression vector is designed.

A number of assays may be carried out using a modified CCR3 receptor of the invention. Initially, assays may be carried out to evaluate the effect of a modification on the CCR3 receptor and to establish whether the modification reduces internalization of a receptor from the cell membrane, the effect on recognition of the phosphorylation sites by kinases and whether the modified receptor is stabilised within the cell membrane. An unmodified version of the CCR3 receptor may be used as a comparison.

Cells of the invention expressing the CCR3 receptor can be used in assays to identify modulators of CCR3 mediated cellular responses and binding to the receptor.. Such assays include ligand binding assays, or functional assays giving output as a result of G protein mediated signalling, for example, by looking at calcium ion influx into a cell. Alternatively, plasma membranes may be prepared from cells expressing CCR3 receptors and assays performed on such membranes. Such assays may be used, for example, to monitor GTPγS binding, for example, using radio labelled ³⁵S GTPγS.

The assays can be used to identify additional ligands which bind to CCR3 receptors. Alternatively, the method can be used to identify agents which antagonize or inhibit binding and responses mediated through a known ligand for CCR3 receptors such eotaxin, eotaxin-2, eotaxin-3, RANTES, MCP-2, MCP-3 or MCP-4. In this assay, a compound under investigation is incubated with cells or membranes containing a modified CCR3 receptor in the presence of the known ligand such as eotaxin. The effect on eotaxin-mediated responses may then be monitored. Control experiments may be run in the presence and absence of eotaxin without a target compound, and in the absence of known ligand such as eotaxin.

In an assay in accordance with the invention, internalization and/or phosphorylation through protein kinase is reduced. This can be done through expression of a modified CCR3 receptor as described above in the cells or membrane under investigation. Alternatively, other methods may be used which modify the activity of the internalization site and/or phosphorylation sites of CCR3 receptors. For example, inhibitors of protein kinase may be added to the assay system to reduce or prevent phosphorylation of CCR3 receptors. Preferably, the reduction in the activity of the phosphorylation sites or internalization sites is carried out using agents which do not otherwise effect the response which is seen on eotaxin binding to CCR3 receptors. Alternatively, identification of specific proteins which bind to the internalization or a phosphorylation site may be used to prevent phosphorylation or internalization of CCR3 receptors.

Agents which modulate CCR3 receptor activity and which have been identified by assays in accordance with the invention can be used in the treatment or prophylaxis of allergic or inflammatory disorders which are responsive to regulation of CCR3 receptor activity. Agents which activate CCR3 receptor activity and/or which have been identified as inhibitors of allergic or inflammatory disease are preferred. Preferably, such agents may be used in the treatment of allergy or asthma as well as opthalmological, inflammatory, gastrointestinal, dermatological, respiratory or pruritic disorders. In particular, such agents may be used in the treatment of conjunctivitis, IBD, eczema, allergic rhinitis, nasal polyposis, atopic dermatitis and pruritis, COPD and other lung disorders and immune disease.

The agents may be formulated with a pharmaceutically acceptable carrier and/or excipient as is routine in the pharmaceutical art. See for example Remington's Pharmaceutical Sciences, Mack Publishing Company, Eastern Pennsylvania 17^{th} Ed. 1985. The carrier or excipient may be an isotonic saline solution but will depend more generally upon the particular agent concerned and the route by which the agent is to be administered.

The agents may be administered by enteral or parenteral routes such as via oral, buccal, anal, pulmonary, intravenous, intra-arterial, intramuscular, intraperitoneal, topical or other appropriate administration routes. A therapeutically effective amount of a modulator is administered to a patient. The dose of a modulator may be determined according to various parameters and especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of adiminstration. Preferably, daily dosage levels are from 5 mg to 2 g.

As noted above the advantages of the invention include the fact that the modified CCR3 receptor may be more readily, consistently and/or adequately expressed in heterologous host cell systems such as mammalian cell systems, especially CHO relative to the unmodified receptor.

### EXAMPLES

PCR primers were constructed to enable amplification of the full length CCR3 receptor as well as mutant constructs. All constructs were tagged with an HA tag for ease of detection using antibodies to the HA.

The following mutations were made:

ΔC- Deletion of C-terminal internalisation signal (YIPF) and all potential C-terminal phosphorylation sites which may be responsible for desensitisation. (Primers NF313/NF311)

Amplification using NF348/311primers results in change of S231 to A231. Ligation of product NF313/NF349 to product NF348/NF311 and reamplification with primers NF313/NF311 results in introduction of mutation S231A into the third intracellular loop and removal of a potential PKC phosphorylation site (which may be responsible for heterologous desensitisation) as well as all of the other C-terminal modifications. Cloned into pCIN6 [Construct ΔC/S231A].

ΔD-Deletion of all potential C-terminal phosphorylation sites; internalisation site retained (primers NF313/NF343)

ΔE-internalisation and one phosphorylation site retained (primers NF313/NF344)

ΔF-only the most C-terminal potential phosphorylation site deleted (primers NF313/NF347)

S231A Same as AC but Ser231 changed to Ala (PKC site 3rd intracellular loop) (NF313/NF349 & NF348/NF311 primers)

Amplification using NF348/311 results in change of S231 to A231. Ligation of product NF313/NF349 to product NF348/NF311 and reamplification with primers NF313/NF311 results in introduction of mutation S231A into the third intracellular loop and removal of a potential PKC phosphorylation site (which may be responsible for heterologous desensitisation) as well as all of the other C-terminal modifications.

F330A. Full length receptor with internalisation signal mutated. Amplification with primers NF345/NF312 results in change of F330 to A330. Ligation of product NF313/NF346 to NF345/NF312 and reamplification with primers NF312/NF313 results in introduction of F330A mutation into the full length receptor. This version of the receptor has the internalisation motif destroyed but retains all the potential phosphorylation sites.

Presence of signal sequence:

Fragments cloned into pCIN6 have addition of N-terminal CD8 (T8) signal sequence and HA tag (pre-fixed with 6 eg 6C).

Fragments cloned into pCIN7 have addition of N-terminal HA tag only (pre-fixed with 7 eg 7C).

The full-length CCR3 receptor was additionally cloned into pCIN6 and pCIN7.

Mammalian Cell culture and transfections.

Transient transfections

HEK293T cells (HEK293 cells stably expressing the SV40 large T-antigen) were maintained in DMEM containing 10% (v/v) foetal calf serum and 2 mM glutamine. Cells were seeded in 60 mm culture dishes and, grown to 60-80% confluency (18-24 h) prior to transfection with pCDNA3 containing the relevant DNA species using Lipofectamine reagent. For transfection, 3 μg of DNA was mixed with 10:1 of Lipofectamine in 0.2 ml of Opti-MEM (Life Technologies Inc.) and was incubated at room temperature for 30 min prior to the addition of 1.6 ml of Opti-MEM. Cells were exposed to the Lipofectamine/DNA mixture for 5 h and 2 ml of 20% (v/v) foetal calf serum in DMEM was then added. Cells were harvested 48-72 h after transfection.

Stable cell line production

A CHO K1 cell was used as a host for subsequent stable transfection of mutated CCR3. In some cases a CHO cell line stably expressing Gα₁₆ was used. Cells maintained in DMEM/F12 supplemented with 2 mM-glutamine and 10% foetal calf serum were transfected using lipofectamine. Each receptor was selected in G418 (1 mg/ml) and then dilution cloned. Individual clones were tested for their ability to elicit a calcium response in the FLIPR in response to stimulation by 100 nM Eotaxin, or to activate GTPγS.

Calcium assays

Cells were plated into black 96 well plates and left to grow to confluence. On the day of assay the cells were loaded with FLUO-3AM (4 μM) in FLIPR buffer (145 mM NaCl, 5 mM KCl, 2 mM CaCl₂, 1 mM MgCl₂, 10 mM HEPES, 10 mM glucose, 3 mM probenicid. pH7.4) and incubated at 37° C. for 1 hour. After this time the cells were washed with FLIPR buffer. Agonists were added using the FLIPR. For each response, peak increase in fluorescence above basal was calculated. The ALFIT model was used to generate concentration response curves. Ionomycin was included as a positive control in each experiment. Antagonists were pre-incubated for 30 mins at 37° C. prior to agonist addition.

Preparation of membranes for GTPγS assay

Plasma membrane-containing P2 particulate fractions were prepared from cell pastes frozen at −80° C. after harvest. All procedures were carried out at 4° C. Cell pellets were resuspended in 1 ml of 10 mM Tris-HCl and 0.1 mM EDTA, pH 7.5 (buffer A) and by homogenisation for 20 s with a polytron homogeniser followed by passage (5 times) through a 25-guage needle. Cell lysates -were centrifuged at 1,000 g for 10 min in a microcentrifuge to pellet the nuclei and unbroken cells and P2 particulate fractions were recovered by microcentrifugation at 16,000 g for 30 min. P2 particulate fractions were resuspended in buffer A and stored at −80° C. until required. Protein concentrations were determined using the bicinchoninic acid (BCA) procedure (Smith et al., 1985) using BSA as a standard.

High affinity [³⁵S]GTPγS binding.

Assays were performed in 96-well format using a method modified from Wieland and Jakobs, 1994. Membranes (10 :g per point) were diluted to 0.083 mg/ml in assay buffer (20 mM HEPES, 100 mM NaCl, 10 mM MgCl₂, pH7.4) supplemented with saponin (10 mg/l) and pre-incubated with 40 μM GDP. Various concentrations of eotaxin were added, followed by [³⁵S]GTPγS (1170 Ci/mmol, Amersham) at 0.3 nM (total vol. of 100 μl) and binding was allowed to proceed at room temperature for 30 min. Non-specific binding was determined by the inclusion of 0.6 mM GTP. Wheatgerm agglutinin SPA beads (Amersham) (0.5 mg) in 25:1 assay buffer were added and the whole was incubated at room temperature for 30 min with agitation. Plates were centrifuged at 1500 g for 5 min and bound [³⁵S]GTPγS was determined by scintillation counting on a Wallac 1450 microbeta Trilux scintillation counter.

Results

Stable cell line

Eotaxin produced a dose dependent increase in Ca++ mobilisation in the stable CHO/G16 cell line expressing CCR3 6ΔC. Pertussis toxin also inhibited the response to eotaxin showing mediation of the Ca++ signalling through Gi heteromeric G proteins.

Eotaxin stimulated GTPγS activity in 3 CHO cell clones expressing the CCR3 6ΔC(FIG. 1). Eotaxin-2 was also active in this assay.

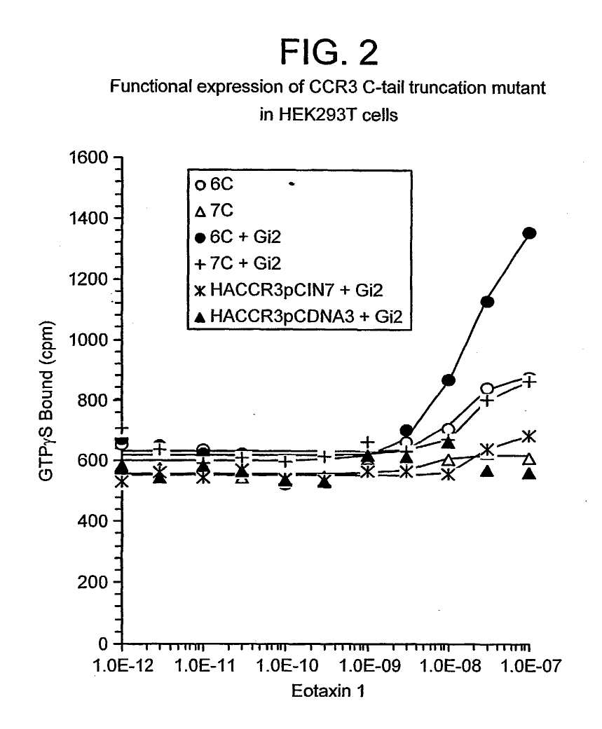

In order to identify the exact residues at the C-terminal tail of CCR3 which preclude its functional expression in cell systems a transient system was established. 6ΔC and 7ΔC CCR3 were expressed transiently in HEK293T cells either in isolation or in combination with Giα2. The results are presented in FIGS. 2, 3 and 4. Exposure of transfected membranes to eotaxin resulted in a significant stimulation of GTP(S binding from cells transfected with 6C and not 7C. Co-expression with Giα2 further enhanced this effect with 6C and allowed a small but significant stimulation in cells transfected with 7C. Eotaxin response was dose-related with a similar EC50 as found in the CHO stable cell line. No eotaxin-mediated stimulation was found in cells co-expressing full length HA-CCR3 and Giα2 in the absence of signal sequence. However, in the presence of a signal sequence, eotaxin-mediated stimulation was seen using the full-length CCR3 receptor, although the maximum response was lower than that seen in 6C.

Alternative mutations of the C-tail (mutants D, E, S231A, F330A) did not have further advantage over 6C truncation, although the 6F mutation was equivalent to 6C.

Hence functional expression of CCR3 in recombinant systems can be achieved by deletion of the C-terminal tail, or deletion of the most C-terminal serine (potential phosphorylation site), or addition of a signal peptide.

1. A modified CCR3 receptor which has been modified to stabilise or enhance expression of the receptor in a cell membrane.

2. A modified CCR3 receptor according to claim 1 which is human receptor which has been modified to stabilise or enhance expression of the receptor in a cell membrane in a heterologous mammalian host cell.

3. A modified CCR3 receptor according to claim 1 or claim 2 wherein the heterologous mammalian host cell is a CHO cell.

4. A modified CCR3 receptor according to any one of claims 1 to 3 wherein the modification comprises deletion or mutation of at least one phosphorylation site in the C-terminal region of the CCR3 receptor.

5. A modified CCR3 receptor according to claim 4 wherein the modification comprises deletion or mutation of at least one phosphorylation site selected from positions 333, 339, 340, 341, 343, 346 and 353 of SEQ ID No. 1 or an equivalent position in a variant receptor.

6. A modified CCR3 receptor according to claim 4 wherein the modification comprises deletion or mutation of the most C-terminal phosphorylation site of CCR3 receptor.

7. A modified CCR3 receptor according to any one of the preceding claims wherein the modification comprises or additionally comprises mutation or deletion of the internalization site YIPF at position 327-330 of SEQ ID No. 1 or an equivalent position of a variant CCR3 receptor.

8. A modified CCR3 receptor according to any one of the preceding claims wherein the modification comprises or additionally comprises an N-terminal signal sequence to enhance delivery of modified receptor to the cell surface.

9. A modified CCR3 receptor according to any one of the preceding claims comprising deletion and/or mutation of at least one phosphorylation site and deletion/mutation of an internalization site.

10. A modified CCR3 receptor according to any one of the preceding claims wherein the modification comprises deletion of the C-terminal region of the receptor at a position to remove all C-terminal phosphorylation sites and the internalization site.

11. A modified CCR3 receptor according to claim 10 wherein the deletion comprises a deletion from position 327 of SEQ ID No. 1 or deletion from an equivalent position of a variant CCR3 receptor.

12. A polynucleotide encoding a modified CCR3 receptor according to any one of the preceding claims.

13. An expression vector comprising a polynucleotide according to claim 12.

14. A host cell transformed with a polynucleotide according to claim 9 or an expression vector according to claim 13.

15. A host cell according to claim 14 which is a heterologous mammalian cell.

16. A host cell according to claim 14 which is a CHO cell.

17. An assay for investigating one or more properties of a CCR3-receptor comprising providing a cell expressing a modified CCR3 receptor according to any one of claims 1 to 11, performing the assay using such cells and investigating thereby one or more properties of the CCR3 receptor.

18. An assay according to claim 17 for identification of modulators of eotaxin-mediated CCR3 receptor activity wherein the assay comprises contacting a compound under investigation with the cell and determining thereby whether a test compound modulates eotaxin-mediated CCR3 receptor activity.

19. An assay according to claim 18 further comprising contacting eotaxin with the test compound and cell under investigation.


SEQUENCE LISTING (2 sequences)

SEQ ID NO: 1 - protein, 355 aa, Homo sapiens [sequence omitted]

SEQ ID NO: 2 - DNA, 1068 bp, Homo sapiens
  CDS (1)..(1068)
atgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggacgaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttag